(19) United States
(12) Patent Application Publication
(10) Pub. No.: US 2016/0271289 A1
(43) Pub. Date: Sep. 22, 2016

(54) Ultraviolet air sanitizing fixture for commercial aircraft

(71) Applicant: William Duffy, Baldwin, FL (US)

(72) Inventor: William Duffy, Baldwin, FL (US)

(21) Appl. No.: 15/062,810

(22) Filed: Mar. 7, 2016

Related U.S. Application Data
(60) Provisional application No. 62/135,892, filed on Mar. 20, 2015.

(51) Int. Cl. A61L 9/20; B64D 13/06

(52) U.S. Cl. CPC A61L 9/20; B64D 13/06; B64D 2013/0603

(57) ABSTRACT

A rod-shaped fluorescent UV light bulb affixed to a socket comprises an ultraviolet (UV) air sanitizing fixture for commercial aircraft, public transit, or heavy equipment. Electric power is provided by the vehicle electrical system. The fixture is installed by insertion into a small circular aperture which has been cut or drilled into the sheet metal of the air circulation ductwork of the vehicle, and securing the socket to the sheet metal.

### CROSS-REFERENCE TO RELATED APPLICATIONS

This application claims the benefit of U.S. Provisional Application No. 62/135,892, filed Mar. 20, 2015, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to transportation equipment and in particular to an ultraviolet air sanitizing fixture for transportation vehicles. The recent concerns about the spread of the Ebola virus have raised awareness of how disease pathogens are spread. The enclosed environment within a transportation vehicle has been a particular target of concern for public health professionals. Theoretically, a highly contagious airborne pathogen could spread from one infected passenger to everyone on board, due to the enclosed environment and the constant circulation of air within the cabin. Fortunately, ultraviolet (UV) light has proven to be effective in killing these airborne pathogens, but the source of the UV light must be shielded to prevent permanent injury to the eyes of the vehicle's occupants.

A search of the prior art reveals various devices which have been developed to cleanse and sanitize the air circulating within vehicles. None are closely related to the present invention, but several include features which resemble those of the present invention. Each has proven to be less than satisfactory in its own way. The present invention has been developed for the purpose of addressing and resolving these disadvantages.

Air sterilizing assembly, U.S. Pat. No. 8,497,491 (priority May 20, 2009), provides an air disinfection device aimed at improving the efficiency and quality of air disinfection or sterilization. The air disinfection device is comprised of a body housing a power supply and control unit, which, in turn, provides an energy storage capacitor, a high-voltage constant current source, an ignition pulse generator, a ferrite-core pulse transformer, and a program control unit. The air disinfection device may also provide an ultra-violet radiation source in the form of a pulsed gas-discharge lamp mounted on the body and enclosed in a tubular quartz casing. Given that the energy storage capacitor and the pulsed gas-discharge lamp may form a discharge circuit connected to the ignition pulse generator through the ferrite-core pulse transformer, the pulsed gas-discharge lamp may be placed in a bactericidal radiation translucent casing, resulting in convection air-cooling due to a natural draft inside the casing that disinfects and sterilizes surrounding air.

Methods and apparatus for sterilization of air and objects, U.S. Pat. No. 8,319,195 (priority Jun. 12, 2003), provides a portable air sterilization apparatus which includes a portable chamber forming an interior volume, a portable source for producing a single beam of collimated light energy, and a rotating beam redirector. The portable chamber includes a first end for introducing air into the chamber, a second end for permitting air to exit from the chamber, a transparent orifice through which a single beam of collimated light energy can be introduced into the interior volume of the chamber; and at least one reflective wall. The rotating beam redirector is adapted to rotate through a complete revolution about a rotational axis and to redirect the single beam of collimated light energy within the chamber during said revolution.

Cabin air quality system, U.S. Patent App. Pub. No. US2005/0053515 (filed Sep. 10, 2003), provides an air quality system comprising at least one air cleaner unit in communication with an interior air space. In one embodiment, an air quality system for an aircraft comprises a first set of air cleaner units in communication with a lower plenum of the aircraft recirculation system and a second set of air cleaner units in communication with an upper plenum of the aircraft recirculation system. Each air cleaner unit comprises at least one adsorbent unit in combination with at least one photocatalytic oxidation unit. In one embodiment, an air cleaner unit comprises a relatively weak adsorbent material upstream from a photocatalytic oxidation unit, and a relatively strong adsorbent material downstream from the photocatalytic oxidation unit. Methods for removing a pollutant from an air stream and for making an air cleaner unit are also disclosed.

Ambient air cleaning, German Patent No. 4318738 (filed Jun. 5, 1993), provides an air cleaning system for cleaning ambient air physically and chemically, conventional filters and/or catalysts are used, using vehicles as the carriers, where the filters and/or catalysts have components not needed for the vehicle operation. After the air has been drawn in and cleaned, it is delivered directly into the vehicle environment.

Air cleaner for vehicle, Japanese Patent No. 2006111227 (filed Oct. 18, 2004), provides an air cleaner for a vehicle capable of eliminating not only odor components floating in the air in a vehicle but also odor components stuck on a wall surface, a seat or the like in the vehicle. The cleaner is provided with an electrostatic atomizing type air cleaning unit having an electrostatic atomizing device for eliminating the odor components by ejecting charged microparticulate water of nano meter size in the vehicle.

Air filtration and purification system for vehicle, U.S. Pat. No. 5,810,896 (filed Jul. 16, 1997), provides an air filtration and purification system for the interior of a closed vehicle, including an outside air intake and an inside air intake. Both of the intakes empty into a common plenum located interior of the vehicle, an air passageway defined by an inlet, in fluid communication with the plenum, and an outlet spaced-apart therefrom for removing the air in the plenum, a plurality of purifying air filters arranged in series in the passageway, a first air movement device interposed the plenum and said passageway inlet to create a flow of air from the plenum to the filters, a second air movement device in the outlet of the passageway to draw said flow of air across the filters, and a vent device in fluid communication with the second air movement device for distributing the filtered air from the second air movement device into the vehicle.

Most of these inventions rely upon mechanical filtration. This creates a disadvantage by requiring the cleaning or changing of the filters. One invention, Japanese Patent No. 2006111227, relies upon a spray of electrostatically charged microdroplets of water, which creates a disadvantage by humidifying the air in an enclosed space, possibly causing discomfort for occupants, corrosion, and other issues. Another invention, U.S. Pat. No. 8,497,491 may provide an ultraviolet pulsed gas-discharge lamp mounted on the body of the device and enclosed in a tubular quartz casing. This creates a disadvantage by creating a risk of unshielded exposure to ultraviolet light among occupants of the vehicle, which may cause permanent eye injuries. The present invention resolves the disadvantages of the prior art and is not anticipated by the prior art.

An ultraviolet air sanitizing fixture for transportation vehicles, which is shielded by being installed within the air circulation ductwork, would resolve this problem.

### SUMMARY OF THE INVENTION

Accordingly, the invention is directed to an ultraviolet (UV) air sanitizing fixture for transportation vehicles. The fixture is comprised of a rod-shaped fluorescent UV light bulb affixed to a socket. Electric power is provided by the vehicle's electrical system. The fixture is installed by insertion into a small circular aperture which has been cut or drilled into the sheet metal of the air circulation ductwork of the vehicle, and securing the socket to the sheet metal.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWING

The accompanying drawing is included to provide a further understanding of the invention and is incorporated into and constitutes a part of the specification. It illustrates one embodiment of the invention and, together with the description, serves to explain the principles of the invention.

FIG. 1 shows a front perspective cutaway view of ductwork with the first exemplary embodiment installed, displaying the fixture 10, the bulb 11, the socket 12, the aperture 13, and the air circulation duct 14.

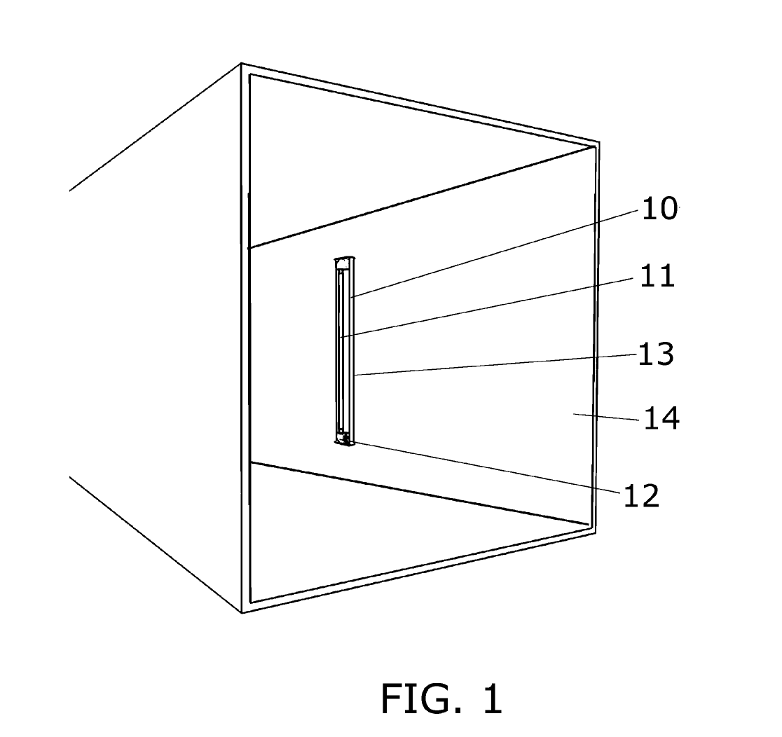

FIG. 2 shows a front perspective cutaway view of ductwork with the first exemplary embodiment not installed, displaying the air circulation duct 14 and the aperture 13.

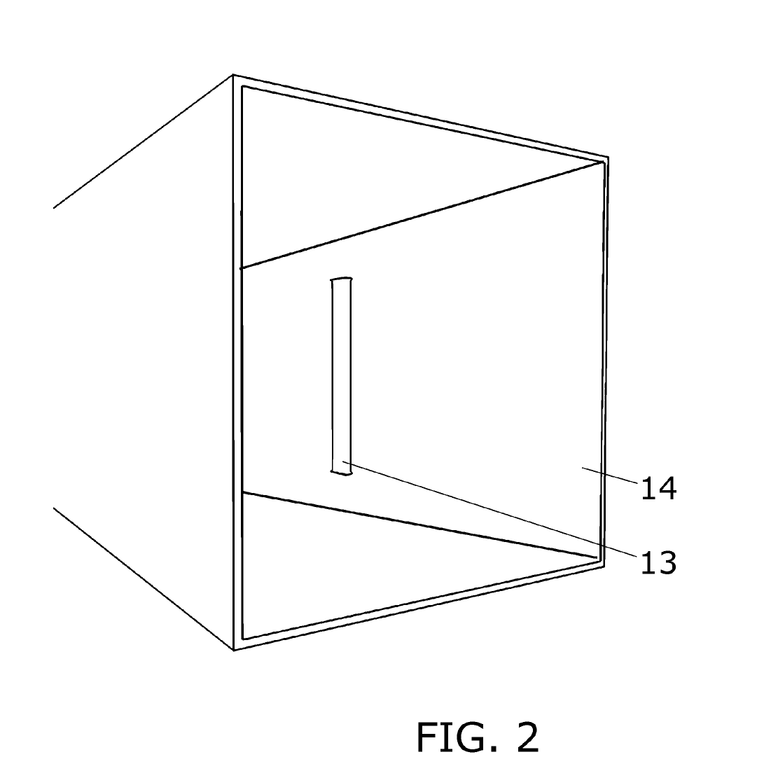

FIG. 3 shows a front perspective cutaway view of ductwork with the second exemplary embodiment installed, displaying the fixture 10, the bulb 11, the socket 12, the aperture 13, and the air circulation duct 14.

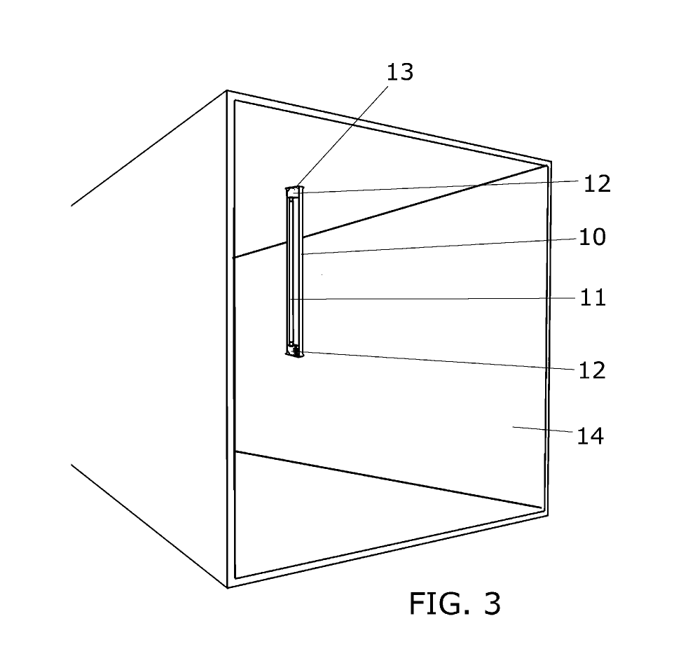

### DETAILED DESCRIPTION OF THE INVENTION

Referring now to the invention in more detail, the invention is directed to an ultraviolet (UV) air sanitizing fixture 10 for transportation vehicles.

It is to be understood that while the invention is intended for installation in transportation vehicles, this is not intended as a limitation. With sufficient modification or with no modification at all, the invention may be installed in any air circulation system where it is desirable to decrease the risk of airborne pathogens, such as in a hospital or in a large construction equipment such as heavy equipment at garbage dumps.

The invention is intended for installation as an after-market modification by the owner of the transportation vehicle, or by a repair or refitting service. An alternate embodiment, which may be installed as original equipment by the manufacturer of the vehicle, is contemplated.

The first exemplary embodiment is comprised of a fixture 10 which provides a rod-shaped fluorescent UV light bulb 11 affixed to a socket 12. Electric power is provided by the vehicle electrical system, and is preferably 120 volts. A circuit breaker may be required. If a 12-volt power supply is provided, a converter may be installed. The fixture 10 is preferably installed by insertion into a aperture 13. The aperture 13 being of the same size and shape as the fixture. This allows the fixture 10 to be placed within one wall of the air circulation duct 14 and lie flat against the wall. The fixture 10 is preferably affixed to the air circulation duct 14 using three screws. Preferably, the bulb 11 is 14 inches in length.

The second exemplary embodiment is comprised of a fixture 10 which provides a rod-shaped fluorescent UV light bulb 11 affixed to a socket 12. Electric power is provided by the vehicle electrical system, and is preferably 120 volts. A circuit breaker may be required. If a 12-volt power supply is provided, a converter may be installed. The fixture 10 is preferably installed by insertion into a small aperture 13. The aperture 13 being, 1.125 inch in diameter, which has been cut or drilled into the sheet metal of the air circulation ductwork 14 of the vehicle, and securing the fixture 10 to the sheet metal with three screws. Preferably, the bulb 11 is 14 inches in length and the fixture 10 is inserted into the aperture 13 at a 90° angle to the wall of the air circulation duct.

To use the first exemplary embodiment, the user activates the air circulation system of the vehicle. The fixture 10 is wired directly to the blower fan, such that when the fan is activated, the fixture 10 is activated as well.

The bulb 11 is preferably manufactured from a rigid, durable material which is transparent, such as glass or plastic. The socket 12 is preferably manufactured from rigid, durable materials such as steel, aluminum alloy, copper alloy, brass, acrylic polymer, and plastic. The wires are preferably manufactured from braided copper alloy wire sheathed in plastic.

Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. An ultraviolet air sanitizing fixture, comprising: a fixture having a socket and providing a fluorescent ultraviolet light bulb affixed to the socket; an air circulation duct; an aperture in said air circulation duct; said fixture being attached to said air circulation duct through said aperture.

2. The ultraviolet air sanitizing fixture of claim 1, wherein the air circulation duct is manufactured from sheet metal.

3. The ultraviolet air sanitizing fixture of claim 1, wherein said fixture is powered by a 120 volt electrical system of a vehicle.

4. The ultraviolet air sanitizing fixture of claim 1, wherein said fixture is powered by a 12-volt power supply and a power converter is installed.

5. The ultraviolet air sanitizing fixture of claim 1, wherein the aperture is 1.125 inch in diameter; said fixture being secured to the sheet metal with a plurality of screws.

6. The ultraviolet air sanitizing fixture of claim 1, wherein the fluorescent ultraviolet bulb is fourteen inches in length and the fixture is inserted into the aperture at a ninety-degree angle to a wall of the air circulation duct.

7. The ultraviolet air sanitizing fixture of claim 5, wherein the fluorescent ultraviolet bulb is fourteen inches in length and the fixture is inserted into the aperture at a ninety-degree angle to a wall of the air circulation duct.

8. The ultraviolet air sanitizing fixture of claim 1, wherein the aperture is equal in size and shape to the fixture; said fixture being secured to the sheet metal with a plurality of screws.

9. The ultraviolet air sanitizing fixture of claim 1, wherein the fluorescent ultraviolet bulb is fourteen inches in length and the fixture is inserted into the aperture such that the fixture lies flat against a wall of the air circulation duct.

10. The ultraviolet air sanitizing fixture of claim 8, wherein the fluorescent ultraviolet bulb is fourteen inches in length and the fixture is inserted into the aperture such that the fixture lies flat against a wall of the air circulation duct.

11. The ultraviolet air sanitizing fixture of claim 1, wherein the fixture is wired directly to a blower fan, such that when the blower fan is activated, the fixture is activated.

12. The ultraviolet air sanitizing fixture of claim 6, wherein the fixture is wired directly to a blower fan, such that when the blower fan is activated, the fixture is activated.

13. The ultraviolet air sanitizing fixture of claim 7, wherein the fixture is wired directly to a blower fan, such that when the blower fan is activated, the fixture is activated.

14. The ultraviolet air sanitizing fixture of claim 9, wherein the fixture is wired directly to a blower fan, such that when the blower fan is activated, the fixture is activated.

15. The ultraviolet air sanitizing fixture of claim 10, wherein the fixture is wired directly to a blower fan, such that when the blower fan is activated, the fixture is activated.

16. The ultraviolet air sanitizing fixture of claim 1, wherein the bulb is preferably manufactured from a rigid, durable material which is transparent, such as glass or plastic.

17. The ultraviolet air sanitizing fixture of claim 1, wherein the socket is preferably manufactured from rigid, durable materials such as steel, aluminum alloy, copper alloy, brass, acrylic polymer, and plastic.

18. The ultraviolet air sanitizing fixture of claim 1, wherein the wires are preferably manufactured from braided copper alloy wire sheathed in plastic.
